(19) United States
(12) Patent Application Publication
(10) Pub. No.: US 2001/0055783 A1
(43) Pub. Date: Dec. 27, 2001

(54) Recombinant phycobiliprotein and phycobiliprotein linker fusion proteins and uses therefore

(76) Inventors: F.C. Thomas Allnutt, Port Deposit, MD (US); Colleen Mary Toole, New Winson, MD (US); John Peter Morseman, Columbia, MD (US)

Correspondence Address: BROBECK, PHLEGER & HARRISON, LLP, ATTN: INTELLECTUAL PROPERTY DEPARTMENT, 1333 H STREET, N.W. SUITE 800, WASHINGTON, DC, 20005, US

(21) Appl. No.: 09/882,093

(22) Filed: Jun. 18, 2001

Related U.S. Application Data
(60) Provisional application No. 60/211,784, filed on Jun. 16, 2000.

(51) Int. Cl. C12Q 1/68; G01N 33/53; G01N 33/537; G01N 33/543; C12P 21/04

(52) U.S. Cl. 435/7.92; 435/6; 530/350; 435/69.7

(57) ABSTRACT

This invention is directed to the utilization of the developing methods for molecular manipulation of cyanobacteria and red algae (and potentially cryptomonad algae) to express of phycobiliproteins and phycobiliprotein linker fusion proteins and their utilization as phycobiliprotein, phycobilisome and subassembly based reagents. In particular, the present invention relates to a method for a specific binding assay to determine a target moiety which is a member of a specific binding pair, and provides an improvement in the method comprising using a detectable label which is a fusion protein containing both a phycobiliprotein domain and another domain corresponding to a first member of a specific binding pair, where the fusion protein binds to a second member of the specific binding pair to provide a detectable labeled complex. The domain derived from the first member of the specific binding pair can be directly fused to the phycobiliprotein or phycobiliprotein linker domain or be separated by a spacer that allows correct folding of both domains.

### CROSS REFERENCE TO RELATED APPLICATIONS

The present application claims priority from U.S. Provisional Patent Application No. 60/211,784 filed Jun. 16, 2000, entitled “Recombinant Phycobiliprotein Fusion Proteins And Uses Therefore.” The disclosure of this application is incorporated, by reference, in its entirety.

### BACKGROUND OF THE INVENTION

1. Field of the Invention

This invention is directed to the utilization of the developing methods for molecular manipulation of cyanobacteria and red algae (and potentially cryptomonad algae) to express of phycobiliproteins and phycobiliprotein linker fusion proteins and their utilization as phycobiliprotein, phycobilisome and subassembly based reagents.

2. Related Art

Phycobiliproteins have been used to fluorescently label one or the other of the members of a specific binding pair by conjugating isolated phycobiliproteins with a specific binding member. These conjugates are available for use in specific analytical procedures. (See U.S. Pat. Nos. 4,520,110; 4,859,582; 5,055,556; and 4,542,104.) Phycobilisomes have also been used to fluorescently label proteins and specific molecules in a manner analogous to that for the phycobiliproteins (See U.S. Pat. No. 5,695,990).

Fusion proteins which have the protein component of a phycobiliprotein subunit(s) have been expressed in E. coli. Fusion proteins having the protein component of a phycobiliprotein subunit have also been expressed in cyanobacterial hosts to produce a functional homologous or heterologous proteinaceous pigment. Proteins fusing a phycobiliprotein domain and a specific binding site domain (e.g. Streptag I, a very small domain having an amino acid sequence of SAWRHPQRGG with a small spacer between cpcB and Streptag I, see Schmidt, et al., (1994) J. Chromatog., 676:337-345) have been expressed in E. coli (these fusion proteins are non-fluorescent, but bind streptavidin) and an algal host (these fusion proteins are normally fluorescent and also bind streptavidin). The resultant fusion proteins were used in situ to probe how structure affects phycobiliprotein formation (C. Toole, Ph.D. Thesis, 1998).

Production of commercial products by expression in recombinant algae has been proposed (specifically, cyanobacterially produced BT), but no commercial products are yet available.

Molecular manipulation of cyanobacteria to express fusion proteins of the phycobiliproteins has been used to express phycobiliproteins in cyanobacteria for use in structural studies, but has not been used for detection technologies.

### SUMMARY OF THE INVENTION

The present invention relates to a method for a specific binding assay to determine a target moiety which is a member of a specific binding pair, and provides an improvement in the method comprising using a detectable label which is a fusion protein containing both a phycobiliprotein domain and another domain corresponding to a first member of a specific binding pair, where the fusion protein binds to a second member of the specific binding pair to provide a detectable labeled complex. The domain derived from the first member of the specific binding pair can be directly fused to the phycobiliprotein or phycobiliprotein linker domain or be separated by a spacer that allows correct folding of both domains. In one embodiment of the invention, the fusion protein contains two or more copies of the domain corresponding to the first member of the specific binding pair. In another embodiment of the invention, the fusion protein also contains a domain corresponding to a member of another specific binding pair, such that the fusion protein is a multivalent, labeled, binding moiety. For phycobiliproteins, phycobilisomes, or subassemblies thereof which have multiple copies of particular phycobiliprotein subunits, the final assembled complex may include both modified (fusion protein) and unmodified subunits.

In one preferred embodiment of the invention, the specific binding assay includes a second fluorescent reagent having a spectrum different from the spectrum of the phycobiliprotein domain, such that, on interaction between the domain corresponding to the first member of the specific binding pair and the second member of the specific binding pair, a detectable change in fluorescence occurs. The detectable change in fluorescence may be quenching of the phycobiliprotein domain fluorescence, or the detectable change in fluorescence is a change in the phycobiliprotein domain fluorescence. Alternatively, the detectable change in fluorescence is a transfer of fluorescent energy from the phycobiliprotein domain to the second fluorescent reagent, resulting in fluorescence emission from the second fluorescent reagent, or the detectable change in fluorescence is a transfer of fluorescent energy from the second fluorescent dye to the phycobiliprotein domain, resulting in fluorescence emission coming from the phycobiliprotein domain. In such a process, the relationship of the fluorescent moieties could reverse to have the phycobiliprotein domain act as a receptor for energy from the second fluorochrome in a manner analogous to that described above.

Immunoassays are examples of the type of use which may be improved by this invention, as are assays for detection of cell specific determinants. The specific binding assay of this invention may be an immunoassay, where the specific binding pair corresponds to an antibody-antigen pair, or the specific binding assay may be a receptor binding assay, or a nucleic acid binding assay, for which the nucleic acid may be DNA, RNA, or protein nucleic acid (PNA). In another embodiment, this invention provides a fusion protein containing a phycobiliprotein and/or phycobiliprotein linker domain and a specific binding domains, the specific binding domain corresponding to a domain of one of a respective ligand or receptor. The receptor may be an antibody domain, biotin binding domain, avidin binding domain, streptavidin binding domain, polyHis binding domain, nucleic acid binding domain, carbohydrate binding domain, lipid or fatty acid binding domain, G protein coupled receptor domain, protein A or G binding domain, a cell surface receptor, or the like, and the ligand may be a hapten, nucleic acid, protein nucleic acid (PNA), hormone, polyamino acid, sugar, fatty acid or lipid, drug, toxin, metabolic byproduct, metabolic cofactor, metabolic intermediate, an antigen or the like. The complex may contain a fusion protein containing a phycobiliprotein and/or phycobiliprotein linker domain which is bound to a cell, organelle, virus or self-replicating particle through non-covalent binding.

In another mode, the present invention relates to methods for biologic activity assays to determine a particular entity which induces a known biologic effect, the improvement comprising using a detectable label which is a fusion protein containing a phycobiliprotein domain and a second domain which undergoes the known biologic effect upon encountering the particular entity, wherein the known biologic effect induces a detectable change in the fusion protein. The biologic activity assay may be an assay for an enzyme, where the second domain of the fusion protein serves as a substrate for an enzyme, such as a phosphokinase or a protease. Alternatively, the biologic activity assay may detect a ribozyme, where the second domain is cleaved by the ribozyme.

In yet another mode, this invention provides a non-covalent complex comprising a first member of a specific binding pair non-covalently bound to a fusion protein containing a phycobiliprotein domain and a specific binding domain corresponding to a second member of the specific binding pair. In one embodiment of this invention, the fusion protein contains two or more copies of the domain corresponding to the second member of the specific binding pair. In another embodiment of this invention, the fusion protein also contains a domain corresponding to a member of another specific binding pair, such that the fusion protein is a multivalent, labeled, binding moiety. In yet another embodiment of this invention, the specific binding assay includes a second fluorescent reagent having a spectrum different from the spectrum of the phycobiliprotein domain, such that on interaction between the two specific binding domains a detectable change in fluorescence occurs. The detectable change in fluorescence may be quenching of the phycobiliprotein domain fluorescence, or the detectable change in fluorescence may be a change in the phycobiliprotein domain fluorescence. Alternatively, the detectable change in fluorescence may be a transfer of fluorescent energy from the phycobiliprotein domain to the second fluorescent reagent, resulting in fluorescence emission from the second fluorescent reagent, or the detectable change in fluorescence is a transfer of fluorescent energy from the second fluorescent dye to the phycobiliprotein domain, resulting in fluorescence emission coming from the phycobiliprotein domain. The specific binding pair may correspond to an antibody-antigen pair, or a receptor-ligand pair, or the specific binding pair may involve specific nucleic acid binding, where the nucleic acid is DNA, RNA, or PNA. In the non-covalent complex of this invention, the fusion protein containing a phycobiliprotein domain may comprise an antibody domain or cell surface domain, a hapten or an antigen domain, or the fusion protein may be bound to a cell, virus, subcellular organelle, prion or biological surface.

In yet another embodiment, this invention provides an isolated, purified fusion protein comprising a domain corresponding to a molecule of at least about 2000 molecular weight and a domain corresponding to a phycobiliprotein and/or phycobiliprotein linker subunit. The molecule may be an avidin binding domain, a streptavidin binding domain, a hormone binding domain, sugar binding domain, drug binding domain, toxin binding domain or an antibody, or a molecule which specifically binds nucleic acid or protein. In an alternative embodiment, the molecule is one not naturally associated with phycobilisomes in algal cells.

In still another mode, this invention provides a method of producing an isolated and purified fusion protein containing a phycobiliprotein domain and a non-phycobiliprotein domain, said method comprising (1) culturing a recombinant cell transformed with a nucleic acid sequence encoding a phycobiliprotein subunit in reading frame with a nucleic acid sequence encoding a polypeptide domain so that the nucleic acid sequences are expressed as a fusion protein; and (2) isolating said fusion protein so that it is separated from all other fluorescent moieties in the cell other than fluorescent moieties which may be part of a spontaneously forming complex containing the fusion protein. Preferably, the recombinant cell is an algal cell, and the nucleic acid sequence encoding the non-phycobiliprotein domain is foreign to the algal cell.

In yet another mode, this invention relates to a method for screening a combinatorial library to determine moieties which recognize a specific target molecule, and the invention provides an improvement comprising using a detectable label which is a fusion protein containing a phycobiliprotein domain and another domain corresponding to the one member of a random peptide library, where the labeled library is used to locate peptide-based domains which bind to the target molecule.

In still another mode, this invention provides a method of producing an isolated and purified fusion protein containing a phycobiliprotein and phycobiliprotein linker domain and a non-phycobiliprotein molecule domain, the method comprising (1) culturing a recombinant cell transformed with a nucleic acid sequence encoding a phycobiliprotein linker in reading frame with a nucleic acid sequence encoding a polypeptide domain so that the nucleic acid sequences are expressed as a fusion protein, wherein said recombinant cell is not an algal cell; (2) isolating said fusion protein, (3) mixing said fusion protein with a plurality of phycobiliprotein subunits or phycobilisome subcomplexes missing the phycobiliprotein linker used in production of the fusion protein, and (4) allowing these to form a complex with the phycobiliprotein linker fusion protein fitting into its proper position in the assembled phycobiliprotein or its subunits and/or phycobilisome or its subassemblies.

In yet another mode, the invention also provides an isolated and purified fusion protein comprising a domain corresponding to a nucleic acid binding domain and a domain corresponding to a phycobiliprotein and/or phycobiliprotein linker subunit. This invention also provides an assay method using the fusion protein or protein complex comprising a domain corresponding to a nucleic acid binding domain and a domain corresponding to a phycobiliprotein and/or phycobiliprotein linker subunit, wherein a synthetic oligonucleotide, having a recognition sequence for the nucleic acid binding domain in the fusion protein, binds to the reagent to form a non-covalent complex containing oligonucleotide bound to fluorescent label. Preferably, the complex is allowed to hybridize to target nucleic acid complementary to the oligonucleotide such that hybridization to the target may be detected.

### DETAILED DESCRIPTION OF THE EMBODIMENTS

As contemplated herein, one amino acid sequence “corresponds” to another amino acid sequence if at least 75% of the amino acid positions in the first sequence are occupied by the same amino acid residues in the second sequence. Preferably 90% of the amino acid positions are identical, and most preferably 95% of the amino acid positions are identical. Alternatively, two amino acid sequences are considered to correspond to each other if the differences between the two sequences involve only conservative substitutions. “Conservative amino acid substitutions” are the substitution of one amino acid residue in a sequence by another residue of similar properties, such that the secondary and tertiary structure of the resultant peptides are substantially the same. Conservative amino acid substitutions occur when an amino acid has substantially the same charge as the amino acid for which it is substituted and the substitution has no significant effect on the local conformation of the protein. Amino acid pairs which may be conservatively substituted for one another are well-known to those of ordinary skill in the art. A first amino acid sequence which corresponds to a second amino acid sequence may have additional peptide sequences attached at one or both ends of the first amino acid sequence without destroying the correspondence so long as the tertiary structure of the first and second sequences are sufficiently similar to confer substantially similar binding properties on the polypeptides containing the sequences. Two polypeptides have substantially similar binding properties if both are capable of binding to the same binding partner, and binding of one polypeptide to the binding partner inhibits binding by the other polypeptide when the two are present in the same solution at concentrations within an order of magnitude.

A “domain” as contemplated herein means a portion of a macromolecule, and a first domain corresponds to a second domain if the topographic and geometric structure of the first domain is substantially similar to that of the second domain, such that the binding characteristics of the two domains are the same. For example, the antigen recognition domain of an Fab fragment corresponds to the antigen binding domain of an immunoglobulin G molecule specific for the same epitope.

A “phycobiliprotein domain” as contemplated herein includes polypeptides substantially similar to any phycobiliprotein subunit or linker, whether intramolecular or intermolecular, as well as complexes of such subunits, including phycobiliproteins, phycobiliprotein discs, phycobiliprotein rods, phycobilisomes or other complexes of such subunits. Phycobiliprotein domains may include all or part of the sequence of a phycobiliprotein subunit, or may correspond to the tertiary structure of a portion of a phycobiliprotein produced by a plurality of fragments of the phycobiliprotein sequence connected by spacer peptide sequences which permit folding to replicate the phycobiliprotein structure. Preferably, phycobiliprotein domains will be associated with prosthetic groups (e.g., chromophores) as necessary for fluorescence. Of course, most phycobiliprotein linker domains would not be expected to have bilin prosthetic groups associated with them. More preferably, phycobiliprotein domains will have tertiary structural characteristics of a phycobiliprotein site or sites for binding phycobilins, and most preferably, amino acids which interact with the phycobilin prosthetic group in a phycobiliprotein are present in equivalent positions of the phycobiliprotein domains.

The term “antibody” encompasses whole immunoglobulins made up of immunoglobulin peptide chains, e.g., two heavy chains and two light chains, as well as immunoglobulin fragments. “Immunoglobulin fragments” are protein molecules related to antibodies, which retain the epitopic binding specificity of the original antibody, such as Fab, F(ab)′₂, Fv, etc.

The “phycobiliprotein fusion” proteins of a phycobiliprotein subunits (α, >or phycobiliprotein linkers (e.g., γ, L_{R}, L_{RC}, L_{R}^{8.9}) could be used as either secondary labels (e.g., phycocyanin fusion with a streptavidin binding site) or in primary detection (e.g., phycoerythrin fusion with a receptor binding domain or specific Fv fragment) either in vivo or in vitro. The advantages this would provide are as follows:

It would provide a fusion protein expressed in an alga either constituitively or under inducer control that, once isolated, can be used directly as a reagent for the detection of a specific binding partner. The expressed fusion protein would be allowed to accumulate within the alga and the proper folding of the phycobiliprotein components allowed. In the native phycobiliprotein production environment, the endogenous lyases will insert the correct chromophores in the correct position to provide fluorescence. The recombinant fusion protein, either with or without its subunit partner(s) or in complexes, will be isolated and, due to post-translational processing in the alga, will have fluorophores correctly and functionally attached. Standard protein purification methods or affinity purification could then be applied to isolate the functional detection reagent directly from the medium or cell lysate.

This method for production of an assay-ready detection reagent (i.e., phycobilisomes decorated with specific binding or recognition sites from the fusion proteins at peripheral rod hexamers, trimers, single subunits, monomers, rods, cores or core-associated substructures) would bypass the traditional requirements for the isolation of the dyes and specific binding partners followed by chemically coupling the dye and binding partner. This would also make the fusion protein-based reagents less modified (therefore more soluble and more like the original binding site, since the non-specific cross-linkers traditionally used could act on chemical moieties that may be important for binding). Chemical modification often damages both the fluor and the binding partner, providing sub-optimal performance of dye and binding agent. Direct isolation of a properly folded binding partner and fluor covalently attached to each other in the recombinant algal strain would bypass this problem. Separate steps for the isolation of pigment, isolation of binding partner, chemical cross-linking and isolation of conjugate can be eliminated, providing improvements in the yields and quality, with a reduction in the labor required for production of these products. Additionally, phycobiliprotein fusions of this type would provide constant and optimal positioning of the fluorescent domain(s) with respect to the binding domain(s) at a constant ratio determined by the genetic construction of the fusion protein and no other external factors. This would deliver a substantially homogeneous reagent (all molecules the same) to provide the highest quality data in terms of consistency and sensitivity of results.

In accordance with this invention, cryogenically preserved algal mutant strains are provided that could be cultured in the light or dark to provide a starting material for purification (e.g., affinity purification) of a specific fusion protein that is properly processed in the alga to add the fluorophores to make it fluorescently labeled, as well as a properly folded binding partner that would recognize the target protein. This would provide an improvement in the cost of manufacturing and also be very efficient due to the lack of chemical modification and harsh isolation procedures. The binding partners could be specific (made in custom fashion for each target) or a generic secondary reagent (e.g., using StrepTag (Schmidt, et al., 1994, J. Chromatog., 676:337-345), streptavidin binding domain, or biotin binding domain (Gomicki, et al., 1993, J. Bacteriol., 175:5268-5272) or anti-species Fv fragment).

The ability to create unknown specificities is also contemplated in this invention. The unique multimeric composition of the phycobiliproteins allows one to make a random set of fusion proteins that can be expressed in the phycobiliprotein subunits such that they did not inhibit the reassociation of the subunits into a multimeric protein. The random mutations could be generated by making random DNA fragments in a DNA sequencer or by other methods known in the art. These fragments would then be made into double-stranded DNA and fused onto the genes for the phycobiliprotein subunits that comprise the phycobiliprotein of interest using standard molecular biological techniques. The genes will be arrayed in such a manner that, when properly expressed and reassembled into functional phycobilisomes, phycobiliproteins or subassemblies, they are displayed on the surface of the phycobiliprotein or clustered in a specified area like that seen for the Streptag expressed in phycocyanin by Toole (1998). The clustered protein fragments will act in concert to form a binding domain. If all the subunits are from a single mutant all the arms of this binding domain will be the same. Diversity can be increased in this instance by dissociating the phycobiliprotein having variations in their fusion tags into subunits, mixing the dissociated subunits in a combinatorial fashion such that a multi-component matrix is formed. The multi-component matrix will then be allowed to reassociate under permissive conditions (e.g. increasing phosphate concentration) and a broad array of potential new receptors will be formed on the surface of the phycobiliprotein fusion protein or complex. Binding assays can be done with these labeled dyes by any number of methods that recognize the target of interest. For instance the target can be coated or covalently attached on plates, then the combinatorially produced fluorescent library added to the wells. The wells that become labeled are then deconvoluted to discover the source strains of recombinant algae and more careful analysis of the possible structure of the receptor can be determined (varying the phycobiliprotein fusion protein subunit composition). Once well documented, the structure of the binding site can be estimated in several ways. The large molecular weight of the complex makes NMR studies less attractive. However, the well described structure of the phycobiliprotein and the known structures of the fusion peptides (after deconvolution) will allow one to mimic the structures being formed in a smaller version such that the structure can be determined by NMR or other standard methods.

It should be noted that a similar approach to making a combinatorial library can be obtained using this invention by expression of a streptavidin binding domain on the phycobiliprotein subunits in a defined position. Then biotinylated fragments of peptide, DNA, RNA or PNA can be attached in a combinatorial fashion and analyzed as described above to find specific binding domains for targets of interest. Other small DNA binding domains known in the literature can also be utilized. An advantage of this is that stable, isotopically labeled biotinylated fragments can be attached to an NMR-silent fusion protein giving an aid to NMR structural determination of the binding site.

Construction of Fusion Protein Vectors

Vectors can be constructed that are useful for production of phycobiliprotein fusions to specific peptides or proteins. Each biliprotein subunit (allophycocyanin α& β, allophycocyanin β^{18.2}, phycocyanin α& β, phycoerythrin α& β and the biliprotein (BP) domain of ApcE) is comprised of 8 α-helices, X-Y-A-B-E-F-G and H connected by irregular loops. (The same system has been described as a 9 α-helix structure with helicies X-Y-A-B-E-F-F′-G and H.) The sequences of these proteins is well documented in the literature (for a review see Apt et al., 1995, J. Mol. Biol., 248:79-96). Helices X and Y form a platform-like domain and the remaining 6-7 helices form the globular domain of the folded biliprotein subunit. Areas for addition or substitution of amino acids within individual phycobiliproteins and phycobiliprotein linkers can be chosen such that they would have minimal impact on the proper folding of the complete protein into its multimeric form while incorporating the added amino acids to provide functionality. Insertion of the binding domain at some sites would prevent incorporation into a complete phycobilisome, while insertion at other sites would allow phycobilisomes to assemble with incorporated fusion proteins to provide additional tools for making novel labeled compounds. The following domains could be used for the construction of chimeric phycobiliprotein subunits and/or linker polypeptides containing useful inserted sequences:

1) The N- and/or C-terminus or region of the previously mentioned proteins,

2) Portions or all of the loop domains between α-helices,

3) Removal of selected domains or portion thereof (corresponding to, e.g., α-helices X, Y, A, B, E, F, F′ and/or G) and subsequent replacement with useful sequences,

4) Addition of useful amino acid sequences at a specific point in a region that will not effect folding of the phycobiliprotein or subunit domain (e.g. the α-helices or loop regions connecting them),

5) Removal of loop domains between helices and subsequent replacement resulting in chimeric polypeptides,

6) Addition of useful amino acid sequences at a loop domain that will not effect folding of the phycobiliprotein or subunit domain.

Phycobiliprotein linker polypeptides can also be molecularly manipulated resulting in a fusion protein, such as:

ApcE—the large phycobilisome core membrane linker polypeptide, biliprotein like-domain mentioned on previous page as site for insertion of useful amion acid sequence, the ARM1 domain for use as separating entity or spacer between two proteins in the fusion constructs discussed above can also be used as a point of insertion of useful amino acids. ARM1, ARM2, ARM3, ARM4 for removal of selected amino acids and substitution with useful sequences, or just addition of amino acids selected with useful sequences.

Rod (L_{R}) and Rod-core Linker (L_{RC}) polypeptides—includes the smaller molecular mass peptides (e.g. group I, the 8 to 10 kDa polypeptides denoted L_{R}^{8.9} in Mastidocladus laminosus).

L_{c}—small capping protein for allophycocyanin, phycocyanin and phycoerythrin

1) removal of the N- and/or C-terminus and replacement with candidate protein resulting in chimera,

2) add domain or entire protein to N- or C-terminus resulting in chimera

Group II includes the larger molecular mass polypeptides of approximately 30 kDa range:

1) removal the of N- or C-terminus and replacement with candidate protein resulting in chimera,

2) add domain or entire protein to N- or C-terminus resulting in chimera.

Alternatively, regions of the protein can be deleted and replaced with residues corresponding to another protein (resulting in a chimera) where the phycobiliprotein linker functions as a scaffold in the resulting assembled biliprotein entity.

Vectors:

A generic vector for construction and manipulation of the genetic constructs for the phycobiliprotein domain/added domain fusion prior to introduction in the alga will have the following components:

a) Insertion site: polycloning or multicloning site for insertion of fusion domain sequences that maintains proper primary amino acid sequence in the resulting chimera. For example if the fusion is to be made at the C-terminus of a phycobiliprotein subunit gene (e.g. cpcB), there would be a polycloning site at the 3′ end of cpcB that would facilitate the cloning of the peptide/protein that is to be fused with the phycobiliprotein subunit at this domain. Several of virtually the same vectors may be used where the position of the polycloning sites will vary depending upon the final construct that is desired. The lipiprotein subunit may be either expressed alone or the corresponding alpha or beta phycobiliprotein subunit gene may be placed into the vector downstream of the phycobiliprotein subunit (e.g. for phycoerythrin where cpeB is fused to protein of interest followed by cpeA in the same reading frame). For an allophycocyanin fusion expression vector, apcA may be upstrean of apcB. Alternatively, the algal gene target may be a linker protein that would not have an associated alpha or beta subunit. Upstream of the multicloning site (before the phycobiliprotein subunit or linker) an inducible promoter may be placed such that addition of some factor will allow expression of the fusion protein (e.g., IPTG can be used for the beta-galactosidase promoter) to confirm its production in the bacterial system prior to its introduction into the algal system. Such a fusion protein expressed in E. coli will not contain the bilins, therefore it will not be fluorescent and will need to be detected by electrophoresis and/or immunoblotting. Proteins expressed in non-algal strains that have not been engineered to contain the bilin production genes could be useful in systems where fusions produced are allowed to self-associate with phycobiliproteins or phycobilisomes extracted from algae lacking that particular subunit or linker thereby generating a complex that contains the desired functionality but is now fluorescently labeled.

b) Promotor region: a strong promoter may be used to drive expression of the fusion constructs in E. coli. Preferably an inducible promoter is better than constitutive promoters since gene products can sometimes be toxic to the cell.

A second vector may be constructed to introduce the fusion protein constructs into the alga at a neutral site (i.e. into a gene that when deleted has no adverse effects on the growth of the alga). Alternatively, the vector described above could be used without a second vector. Currently, genetic systems are most extensively documented for cyanobacterial or bluegreen algal systems. However, eukaryotic algae could also be utilized in a similar manner. The red algae have been genetically manipulated, and their genetic systems are still in development, as are cryptomonad algae genetic systems. Discussion here will focus on cyanobacterial systems but could apply to the other algae. This second vector would include many of the elements outlined above, including integration of the gene of interest into the genome of the cyanobacterium, which would be facilitated by being flanked on both sides by cyanobacterial genomic DNA corresponding to a neutral gene. An antibiotic gene (different from the one used to create the null mutant geneotype) would reside in the vector between the two flanking regions of the neutral gene as would also a polyclonal site for the insertion of the gene of interest (i.e. fusion protein DNA).

Libraries Expressing Random Peptides on a Phycobiliprotein Subunit or Phycobiliprotein Linker Polypeptide.

The ability to create previously unknown specificities is also contemplated in this invention. The unique multimeric composition of the phycobiliproteins allows one to make a random set of fusion proteins that can be expressed in phycobiliprotein subunits and linkers such that they do not inhibit the reassociation of the subunits into a multimeric protein complex. The many mutations can be generated by making random DNA fragments in a DNA sequencer. These fragments can then be made into double stranded DNA and fused onto the genes for the phycobiliprotein α and/or β subunits and/or linkers that comprise the gene(s) of interest (contemplates possible multiple fusions in separate subunits and/or linkers). The genes will be arrayed in such a manner that, when properly expressed and reassembled they are displayed on the phycobiliprotein surface or clustered in a certain area like that seen for the Streptag I expressed in phycocyanin by Toole (1998). The clustered protein fragments will act in concert to form a binding domain after assembly of the phycobilisome or substructures thereof. If all the fusion proteins are from a single mutant, all the members of this binding domain will be the same functionality.

Diversity can be increased in this instance by dissociating the fusion proteins into their individual subunits, then mixing the disassociated subunits in a combinatorial fashion such that upon reassembly a multi-component matrix is formed. If non-fluorescent linker fusion proteins are used, they can be grown in E. coli or other heterotrophic expression systems, purified then followed by reassociation with dissociated phycobilisome components at premissive conditions to allow association into a complex containing the fusion protein linkers from E. Coli. The multi-component matrix may then be allowed to reassociate under permissive conditions such that a broad array of new receptors will be formed. Use of multiple types of subunit fusion proteins (e.g., both α and β) may further increase diversity.

Binding assays can be done with these labeled dyes by any number of methods that recognize the target of interest. For instance, the target protein can be passively coated on a plate, then the combinatorially produced fluorescent fusion protein library added to the wells. The wells that become fluorescently labeled are then deconvoluted to discover the source strains of recombinant algae and more careful analysis of the possible structure of the receptor can be determined (varying the phycobiliprotein or phycobilisome fusion protein subunit composition for binding optimization).

Once well documented binding acitivity is identified the structure of the binding site can be estimated in several ways. The large molecular weight of the complex makes NMR studies less attractive. However, the well described structure of the phycobiliprotein and the known structures of the peptides (after deconvolution) will allow one to mimic the structures being formed in a smaller version that can be determined by NMR or other standard methods.

It should be noted that a similar approach to making a combinatorial library can be obtained using this invention by expression of streptavidin on the phycobiliprotein subunits or phycobiliprotein linkers in a defined position. Then biotinylated fragments of peptide, DNA, RNA or PNA can be attached in a combinatorial fashion and analyzed as described above to find specific binding domains for targets of interest.

This could also be used in a fluorescence resonance energy transfer (FRET) type system. The fusion proteins could be made to different subunits and/or linkers such that one will donate energy to another to give fluorescence at a different wavelength on binding of the specific binding pair in the absence of competitive substances. For instance, the allophycocyanin alpha subunit could be modified to express a receptor domain and the R-phycoerythrin alpha subunit could be made to express a peptide that mimics the target. In the absence of competitor these would bind to each other and give emission of allophycocyanin (650 nm) on excitation of R-phycoerythrin (488 nm). In the presence of competing analyte, one would get emission at 573 nm. This provides a homogeneous assay for the target.

Modifications of Subunit Interfaces to Obtain Novel Spectral Characteristics

The use of multiple fluorochromes for detection of multiple analytes is a growing alternative to sequential analysis of various antigens. Multicolor or multiplexed assays are finding application in many different areas such as 1) flow cytometry (Schmidt, I., 2000, J. Immunol. Meth., 235, 121-131), 2) microplate assay, 3) DNA hybridization/chromosome mapping (Garini, et al., 2000, U.S. Pat. No. 6,066,459), 4) immunoblot detection (Gingrich, 2000, Biotechniques, 29:636642), 5) biochips and arrays (Lievan, BA, 2000, US Pat. No. 6,097,458, and 6) gene expression (Dunnington & Moore, 1998, PCT Pub. No. WO 98/48274). In flow cytometry the need for new fluorescent dyes is accute as they try to produce more data out of each run to compete with array technology in the detection race. The use of three and four color flow cytometry has become routine. However, 8 to 12 color detection has also been reported as researchers try to redefine what is possible and use flow cytometers as liquid arrays (Taylor, et al, 2001, Biotechniques, 30:661669).

New fluorescent properties can be obtained by molecular modification of subunit interfaces resulting in subunits that no longer bind to their class-specific partners and instead bind to subunits of another class (i.e. PCα/PEβ, PEα/PCβ, PECβ/APα or any other possible combination). Typically, phycocyanin beta subunits bind only with phycocyanin alpha subunits and phycoerythrin alpha subunits bind only with phycoerythrin beta subunits. However, by changing specific amino acids comprising the αβ interface (that are class-specific) to isologous residues from other biliprotein classes (using site directed mutagenesis), it is feasible to have phycocyanin alpha subunits bind with phycoerythrin beta subunits, or have any other possible combination of subunits binding to each other. This can result in altered spectral properties for biliprotein monomers thereby expanding the range of fluorescent reagents possible by this invention.

Another potential site for molecular manipulation is the small linker protein of allophycocyanin (L_{C}^{7.8}). The crystal structure for allophycocyanin and this small linker protein has been elucidated (Reuter, Wolfgang, et. al., 1999, Proc. Natl. Acad. Sci. USA, 96:1363-1368). A large sequence database is also available, enabling molecular manipulation of these two proteins (site-directed mutagenesis) ultimately resulting in altered spectral properties for allophycocyanin. This small linker polypeptide L_{C}^{7.8} consists of a three-stranded β-sheet and two α-helices connected by a random coil. The linker is predominantly located between two β-subunits where it directly interacts with their chromophores. The N-terminal domain of the longer α-helix of the linker interacts with the 2^{nd} monomer of the trimer. A second linker chromophore interaction is between charged and polar residues in the loop between β strand 1 and the short α-helix of the linker polypeptide with monomer 1. The distinct interactions between specific amino acids of the linker and an allophycocyanin monomer give a starting point for site-directed modifications that can result in altered spectral properties for allophycocyanin fusion products.

### EXAMPLES

In order to facilitate a more complete understanding of the invention, a number of Examples are provided below. However, the scope of the invention is not limited to specific embodiments disclosed in these Examples, which are for purposes of illustration only.

### Example 1

Production of a Phycocyanin Alpha Subunit-spacer Fusion Protein Reagent that can be used for the Specific Detection of Human Chorionic Gonadotropin (hCG).

Using a construct produced as described in the previous section that contains an endogenous promoter for the phycocyanin operon wherein the alpha subunit gene (cpcA) has been fused with a spacer region and a specific binding region of human chorionic gonadotropin. In this example, the construct has been transformed into a cyanobacterial mutant strain that is a null mutant of the cpcA gene (Synechocystis sp. PCC 6803); this mutant is naturally transformable by allowing the cyanobacterial cells to adsorb the DNA construct naturally as described in Bryant et al. Molecular Biology of the Cyanobacteria (1994 Kluwer Academic Publishers). The cells so transformed will incorporate a functional cpcA fusion into the null mutant and express a modified CpcA-spacer-hCG receptor that will accumulate as unassembled subunits and/or be incorporated into the natural trimer forming an (αβ)₃ complex structure that can be used alone or as part of a final phycobilsome or subassembly for receptor binding assays.

Alternatively, the construct produced as described in the previous sections where the binding region of hCG has been fused to cpcA can be transformed into a wild-type cyanobacterium where both fusion phycobiliprotein subunits and wild-type subunits would form functional phycobilisomes and some unassembled (monomeric, trimeric and hexameric) complexes. Based upon previous experiments (Toole, 1998), the fusion proteins would assemble preferentially at the most distal position on the phycobilisome rod sub-structures and the wild-type subunits would assemble closer to the core. This would result in a phycobilsome decorated with functional binding domains at its periphery.

Such a mutant will now form fusion proteins or subunit fusions that can be used either as is or assembled in vitro into larger complexes. The mutant will be grown up in large photobioreactors in the presence or absence of selective antibiotic(s) (depending on the construct used). Some cyanobacteria can also be grown heterotrophically or mixotrophically in a fermentor. A continuous centrifuge or other type of harvesting equipment will be used to remove the cells from the medium. Cells will then be broken either by cavitation, grinding or shearing and the fusion protein complex extracted in aqueous buffer containing phosphate (100 mM sodium phosphate pH 7.0) from the cell lysis materials. High phosphate conditions would be required if phycobilisomes are to be the final reagent produced by this method (i.e. 0.75 M potassium phosphate). The cell debris is removed by centrifugation and/or filtration, and then the fusion protein purified by an affinity column containing hCG immobilized to resin. This resin could be utilized multiple times for economic reasons. Alternatively, non-affinity purification could be utilized. Methods commonly employed for phycocyanin purification could also be applied with some modification depending on the fusion protein. A hydroxyapatite column would be equilibrated in low phosphate buffer, then the clarified cell debris loaded onto the column. The phycocyanin fusion would bind to the column and other materials would be removed. A gradient of phosphate concentrations would be applied to remove the fusion protein in a discrete band. The purified fusion would then be further purified on a size exclusion column to obtain a final purified reagent. The reagent could then be freeze dried with a cryoprotectant, such as sucrose or trehalose, for optimal storage. The reagent would be resuspended and used directly in specific binding reactions for the detection of hCG.

A typical binding reaction would be where a microplate well had immobilized on it hCG (either covalently or via a specific monoclonal antibody that did not recognize the same binding site as the receptor expressed in the fusion). Then the sample containing hCG to be measured will be added to the well either simultaneously or sequentially with the phycocyanin-spacer-hCG fusion protein. The amount of fluorescent fusion product binding to the plate will be inversely proportional to the amount of hCG in the sample.

### Example 2

Production of a Phycocyanin Alpha Subunit Fusion Protein Reagent that can be used for the Specific Detection of Human Chorionic Gonadotropin.

Same as above but using a construct that does not contain a spacer region between the cpcA and hCG receptor peptide.

### Example 2b

Production of a Phycocyanin Beta Subunit Fusion Protein Reagent that can be used for the Specific Detection of Human Chorionic Gonadotropin

Same as above except construct would be transformed into a cyanobacterium mutant strain that has a knock out of cpcB. This example also encompasses the introduction of this construct into wild-type cyanobacterium. The construct of this example may or may not contain a spacer between cpcB and hCG receptor.

### Example 3

Production of an Allophycocyanin Beta Subunit Fusion Protein Reagent that can be used for Specific Detection of Biotin.

In a method analogous to that used for the cpcA method described above, apcB is modified to be expressed as a fusion protein containing a spacer region between it and a monomeric streptavidin binding site. The ARM1 domain of apcE is a good example of a possible spacer domain that will allow normal folding of the phycobiliprotein domain as well as proper folding of the inserted domain. The monomeric streptavidin-binding site could either be fused to the carboxyl or amino terminus of the biliprotein subunit. The construct is transformed into a red alga using microbiolistics methods and a recombination event allowed to proceed whereby either an apcB null mutant is replaced with the construct or an endogenous gene is replaced with the modified gene fusion or the modified apcB is placed into a wild-type background. The mutants are selected for the expression of fusion protein either by selection for allophycocyanin production (when using an allophycocyanin null mutant this is possible) or by biotin binding of expressed fusion protein. This could be done looking for fluorescence energy transfer (FRET) type reactions with Eu³⁺as described by Park et al (1999, Anal. Biochem. 269, 94-104) for analogous synthetically produced reagent. This utilizes a Eu³⁺recognition of a streptavidin whereby binding to the receptor brings the Eu³⁺within a permissive distance for fluorescence resonance energy transfer. The cells having this fusion product would then fluoresce red on Eu³⁺excitation in a time-resolved manner. The mutants expressing the correct fusion product would then be cultured, harvested and broken as described above for phycocyanin fusion product. The clarified lysis buffer would be passed over an iminobiotin column such that fusion proteins expressing streptavidin monomers would be bound to the column. The imminobiotin would then allow one to collect the fusion protein by treatment with 50 mM ammonium acetate (pH 4.0) plus 0.5 M NaCl or 1.0 M acetic acid. This material could then be used in the same assay described by Park et al. (1999) as a reagent for high throughput screening assays.

### Example 3b

Production of an Allophycocyanin Alpha Subunit Fusion Protein Reagent that can be used for Specific Detection of Biotins

Same as the above example but using the apcA gene in place of the apcB gene. To assure better binding a random sequence can be placed as a spacer between the streptavidin binding site and the apcB gene.

### Example 4

Random Peptide Libraries Produced using apcE Gene Fusions.

Genes encoding random peptides can be generated using the methods of Anderson et al., 2000 (See PCT Publ. No. WO 00/20574). These can then be fused to the apcE gene of Synechococcus sp. (in the B-E loop region of the biliprotein like domain) such that expression does not interfere with its assembly. The constructs may then be transformed into an apcE null mutant of the bluegreen alga using electroporation as described in the art (alternative methods for transformation are naturally competant cells, microbiolistics, triparental mating). Thiel T, J. Bacteriol., 171(1):5743-6 (1989). Transformed cells may be identified by the assembly of allophycocyanin into the phycobilisome. Cultures may be initiated and sequential affinity isolations of the apcE fusions obtained using an anti-allophycocyanin polyclonal antibody column to capture all of the fusion proteins expressed in each clonal strain. Each clonal strain represents a separate random peptide fusion. These isolated allophycocyanin-peptide fusions are then arrayed into a library on 96 well plates to provide sources of random peptides labeled with fluor. Alternatively, proteins from genes encoding random peptides fused to the B-E loop domain of ApcE may be produced in E. coli. These ApcE-random peptide fusions may then be assembled into functional fluorescent complexes in vitro by mixing ApcB and ApcA subunits with the recombinant proteins produced in E. coli. These fluorescent complexes can be purified away from unassembled subunits by using methods described above.

### Example 5

Orphan Receptor Screen Utilizing apcE Gene Fusions to Random Peptides.

An orphan receptor is coated onto a microplate using covalent or non-covalent means. These are washed repeatedly with PBS then the apcE-random peptide library plates are used as a source plate to introduce the labeled random library into the receptor bound plates. The reagents are allowed to react together for 30 min with gentle agitation, then the plates washed three times with PBS buffer. Fresh buffer is added to the plates and the plates read wet on a fluorescent or visible plate reader (fluorescent giving better sensitivity). Random peptides that bind to the specific receptor are detected by an increase in fluorescence. The source plate allows the investigator to go back to the original clone and isolate the apcE-peptide fusion by affinity methods previously described. The random peptide is cleaved at the cleavage site by the proper enzyme, and the fluorescent ApcE separated from the peptide by size-exclusion chromatography using SEPHACRYL S100 resin. This peptide is then sequenced using standard methods to describe the specific peptide that binds to the receptor.

### Example 6

Specific Reagent for use in Detection of Orphan Receptor.

Utilizing the above method, a reagent is isolated that can specifically detect the orphan receptor. This can then be used to localize the receptor in the tissue of origin to get an idea of where an unknown receptor is located and lead to eventual elucidation of its function. The tissues may need to be fixed and permeabilized in order for these large fusion proteins to access the target antigens. This can all be done quickly with little or no information on the structure of the receptor or its function.

### Example 7

Combinatorial Application.

A combinatorial library of DNA sequences encoding peptides is produced by known methods and fused as described above into the phycobiliprotein or phycobiliprotein linker. This library of labeled fusion proteins is then used as an affinity reagent to select for the tags with affinity for specific targets. The targets will be labeled with the peptide/phycobiliprotein fusion and therefore selectable. The random peptide library will then be mined for the proper peptide to use in, e.g., drug discovery.

### Example 8

Reassociation of Linker with Holoprotein

A phycobiliprotein linker polypeptide from a cyanobacterium or red alga is produced in E. coil using known sequences such that a specific sequence for recognition of a component, such as an antibody or receptor, is fused to the section of the linker polypeptide that does not associate with the phycobiliprotein (minus any associated linker) or phycobilisome. This linker polypeptide fusion may be purified to homogeneity (or not). At this point the recombinant peptide could be labeled with a heterobifunctional cross-linker to add a reactive group such as an azido group which could be used to make the spontaneously reassociated linker/phycobiliprotein covalently stabilized. Alternatively, the reassociated linker/phycobiliprotein complex can be left without further stabilization. The linker (stabilized or native) is then added to the phycobiliprotein (minus associated linker) and allowed to reassociate under permissive conditions (e.g., high phosphate). This will result in a modified phycobiliprotein or complex having a recombinant linker attached to it and available for interaction with its specific binding pair or target molecule. If an azido modified linker was used, it can be covalently attached to the target molecule by actinic light.

### Example 9

Fusion of a Tyrosine Kinase Recognition Motif to the Carboxy- or Amine-terminus of the Small Linker Protein CpcD.

An antibiotic resistance marker to which the wild type algal strain is sensitive will be used to interrupt the endogenous cpcD creating a CpcD null mutant cyanobacterium which can then be transformed with a cpcD fusion construct into a neutral part of the cyanobacterial genome. The motif fused to the carboxyl or amino terminus of CpcD is an efficient substrate for in vitro phosphorylation by tyrosine protein kinases. The fluorescent complexes will be purified from cyanobacteria using techniques described earlier and consist of a fluorescent complex (phycobilisome) that has small peptides (kinase substrates) extending outward from each rod sub-structure that are capable of being phosphorylated by proteins kinases. Homogeneous FRET-based assays can then be used to determine tyrosine kinase activity or the effect of inhibitors on phosphorylation.

### Example 10

DNA Binding Complex.

In a method analogous to that used for the cpcA method described previously, apcB is modified to be expressed as a fusion protein containing a DNA binding site. This monomeric DNA-binding site could either be fused to the carboxy or amino terminus of the biliprotein subunits. The product is transformed into an alga using electroporation, natural uptake, tirparental mating or microbiolistics methods and a recombination event allowed to proceed whereby either an apcB null mutant specifically complemented with the DNA-binding domain apcB-fusion construct or an endogenous gene is replaced with the modified gene. The mutants are selected by the expression of fusion protein either by selection for allophycocyanin production (null mutant) or by DNA binding of fusion protein expression. This can be done looking for FRET type reactions with Eu³⁺as described by Park et al. (1999, Anal. Biochem. 269, 94-104) for analogous synthetically produced reagent. This application utilizes a Eu³⁺labeled oligonucleotide containing the DNA sequence recognized by the fusion protein's DNA binding domain whereby recognition of the DNA sequence by the fusion protein brings the Eu³⁺within a permissive distance for fluorescence resonance energy transfer. The cells having this fusion product will then fluoresce red on Eu³⁺excitation in a time-resolved manner. To make the reagent, the mutants expressing the correct fusion product will then be grown up, harvested and broken as described above for phycocyanin fusion product. Allophycocyanin isolation may be accomplished using standard protein isolation protocols involving ammonium sulfate precipitation at 65% saturation, dialysis, separation on hydroxylapatite and final clean up by gel filtration.

### Example 11

Homogeneous Assay Based on Disruption of Energy Transfer from a Phycobiliprotein Fusion Protein to another part of the same Complex or an Associated Subunit.

A fusion protein is engineered as described in this invention to include an amino acid sequence that is a substrate of a particular enzyme of interest, such as a protease, whose action on the amino acid sequence will either cause a breaking of the amino acid backbone, dissociation of phycobilisome complex or a conformational change resulting in an increase or a decrease in fluorescence energy transfer or quenching. The presence of the target enzyme will then be monitored by measuring the appearance of a new fluorescence emission (in the case of where energy transfer resulted in quenching of signal) or the decrease in acceptor fluorescence accompanied by an increase in donor fluorescence (in the case of a FRET pair). This can be formatted in many different ways; a description of two approaches is provided below.

The apcE gene, encoding the allophycocyanin specific linker polypeptide that functions in assembly of the allophycocyanin core complex, can be modified so that the small loop-like structures (e.g., ARM1, ARM2, ARM3, and ARM4) exposed on the surface of the phycobilisome core complex contain the sequence—Ile-Glu-Gly-Arg-X- either in single or multimeric form. Upon expression in an algal strain having phycocyanin and allophycocyanin comprising its phycobilisome, the modified apcE gene would function to assist in assembly of the core complex having exposed regions that are susceptible to proteolytic action by Factor Xa (coagulation Faction Xa). This isolated reagent will provide emission at 666 nm until it is destabilized by the action of Factor Xa on the exposed amino acid sites, whereby a shift to the blue would occur, resulting in emission at 640 nm or below (i.e., phycocyanin is destablized from allophycocyanine, and allophycocyanin is destabilized to trimers or monomers).

An alternative approach would be to provide a binding site for a donor fluor (e.g., R-phycoerythrin) on the fusion protein as well as a protease cleavage site. One example of this could be engineering an apcA gene to contain both a thrombin cleavage site (containing an exposed arginine) and a Fv fragment which recognizes the phycoerythrin β subunit. The fusion protein reagent would be isolated on an affinity column where R-phycoerythrin has been immobilized by a cleavable linker. The fusion protein Fv region would bind to the CpeB portion of the bound R-phycoerythrin. The proteins bound to the resin would be washed to remove unbound material. The column would then be treated to cleave the fusion protein/R-phycoerythrin reagent and concentrate it in the elution medium. The reagent would be dialyzed to remove cleavage materials and then be assay-ready. In such an assay, one would detect the presence of thrombin activity by a decrease in 650 nm emission from allophycocyanin on excitation of the R-phycoerythrin (e.g. at 488 nm from an argon laser) as the FRET pair (APC acceptor, R-PE donor) is disrupted by the protease. This could also be measured by an increase in 573 nm emission of the R-phycoerythrin that can no longer transfer energy to allophycocyanin. A ratiometric analysis of both 650 and 573 nm emissions could provide an enhancement of signal and internal control.

### Example 12

Homogeneous Assay for DNA Hybridization using two Phycobiliprotein Fusion Proteins.

As described in the invention two fusion proteins are designed with streptavidin binding pockets (that specifically bind to biotin), the fusion proteins being components of dyes that can act as a donor or acceptor pair. Alternatively, one of these fusions can be used with any streptavidin or avidin labeled fluor that can act as part of a donor or acceptor pair. In the case of two fusion proteins from this invention, the phycobiliprotein fusions are incorporated into intact phycobiliproteins and the modified phycobiliproteins isolated. Separately the fusion proteins are allowed to bind respectively to two biotin labeled complementary pieces of DNA, RNA or PNA. These two reagents, when combined in hybridization buffer at appropriate stringency, will hybridize and form an energy transfer pair that will quench donor fluorescence and allow emission at the acceptor fluorescence. The presence of complementary nucleic acid in the sample being analyzed would compete with the hybridization of the donor/acceptor pair, resulting in decreased emission of the acceptor in proportion to the amount of the complementary (target) nucleic acid in the sample.

Although the foregoing invention has been described in some detail by way of illustration and example for purposes of clarity of understanding, it will be obvious that certain changes and modifications may be practiced within the scope of the appended claims. Modifications of the above-described modes for carrying out the invention that are obvious to persons of skill in medicine, immunology, fluorescent labeling, recombinant DNA, and/or related fields are intended to be within the scope of the following claims.

All publications and patent applications mentioned in this specification are indicative of the level of skill of those skilled in the art to which this invention pertains. All such publications and patent applications are herein incorporated by reference to the same extent as if each individual publication or patent application was specifically and individually indicated to be incorporated by reference.

1. In a method for a specific binding assay to determine a target moiety which is a member of a specific binding pair, the improvement comprising using a detectable label which is a fusion protein containing a phycobiliprotein domain and another domain corresponding to a first member of a specific binding pair, wherein the fusion protein binds to a second member of the specific binding pair to provide a detectable labeled complex.

2. The method of claim 1, wherein the fusion protein contains two or more copies of the domain corresponding to the first member of the specific binding pair.

3. The method of claim 1, wherein the fusion protein also contains a domain corresponding to a member of another specific binding pair, such that the fusion protein is a multivalent, labeled, binding moiety.

4. The method of claim 1, wherein the specific binding assay includes a second fluorescent reagent having a spectrum different from the spectrum of the phycobiliprotein domain, such that, on interaction between the domain corresponding to the first member of the specific binding pair and the second member of the specific binding pair, a detectable change in fluorescence occurs.

5. The method of claim 4, wherein the detectable change in fluorescence is quenching of the phycobiliprotein domain fluorescence.

6. The method of claim 4, wherein the detectable change in fluorescence is a change in the phycobiliprotein domain fluorescence.

7. The method of claim 4, wherein the detectable change in fluorescence is a transfer of fluorescent energy from the phycobiliprotein domain to the second fluorescent reagent, resulting in fluorescence emission from the second fluorescent reagent.

8. The method of claim 4, wherein the detectable change in fluorescence is a transfer of fluorescent energy from the second fluorescent dye to the phycobiliprotein domain, resulting in fluorescence emission coming from the phycobiliprotein domain.

9. The method of claim 1, wherein the specific binding assay is an immunoassay, said specific binding pair corresponding to an antibody-antigen pair.

10. The method of claim 1, wherein the specific binding assay is a receptor binding assay.

11. The method of claim 1, wherein the specific binding assay is a nucleic acid binding assay.

12. The method of claim 11, wherein the nucleic acid is DNA.

13. The method of claim 11, wherein the nucleic acid is RNA.

14. The method of claim 11, wherein the nucleic acid is protein nucleic acid (PNA).

15. In a method for biologic activity assays to determine a particular entity which induces a known biologic effect, the improvement comprising using a detectable label which is a fusion protein containing a phycobiliprotein domain and a second domain which undergoes the known biologic effect upon encountering the particular entity, wherein the known biologic effect induces a detectable change in the fusion protein.

16. The method of claim 15, wherein the biologic activity assay is an assay for an enzyme, and the second domain serves as a substrate for the enzyme.

17. The method of claim 15, wherein the enzyme is a phosphokinase.

18. The method of claim 15, wherein the enzyme is a protease.

19. The method of claim 15, wherein the biologic activity assay detects a ribozyme, and the second domain is cleaved by the ribozyme.

20. A non-covalent complex comprising a first member of a specific binding pair non-covalently bound to a fusion protein containing a phycobiliprotein domain and a specific binding domain corresponding to a second member of the specific binding pair.

21. The complex of claim 20, wherein the fusion protein contains two or more copies of the domain corresponding to the second member of the specific binding pair.

22. The complex of claim 20, wherein the fusion protein also contains a domain corresponding to a member of another specific binding pair, such that the fusion protein is a multivalent, labeled, binding moiety.

23. The complex of claim 20, wherein the specific binding assay includes a second fluorescent reagent having a spectrum different from the spectrum of the phycobiliprotein domain, such that on interaction between the two specific binding domains a detectable change in fluorescence occurs.

24. The complex of claim 23, wherein the detectable change in fluorescence is quenching of the phycobiliprotein domain fluorescence.

25. The complex of claim 23, wherein the detectable change in fluorescence is a change in the phycobiliprotein domain fluorescence.

26. The complex of claim 23, wherein the detectable change in fluorescence is a transfer of fluorescent energy from the phycobiliprotein domain to the second fluorescent reagent, resulting in fluorescence emission from the second fluorescent reagent.

27. The complex of claim 23, wherein the detectable change in fluorescence is a transfer of fluorescent energy from the second fluorescent dye to the phycobiliprotein domain, resulting in fluorescence emission coming from the phycobiliprotein domain.

28. The complex of claim 20, wherein the specific binding assay is an immunoassay, said specific binding pair corresponding to an antibody-antigen pair.

29. The complex of claim 20, wherein the specific binding assay is a receptor binding assay.

30. The complex of claim 20, wherein the specific binding assay is a nucleic acid binding assay.

31. The complex of claim 30, wherein the nucleic acid is DNA.

32. The complex of claim 30, wherein the nucleic acid is RNA.

33. The complex of claim 30, wherein the nucleic acid is protein nucleic acid (PNA).

34. The complex of claim 20, wherein the fusion protein containing a phycobiliprotein domain comprises an antibody domain or cell surface domain.

35. The complex of claim 20, wherein the fusion protein containing a phycobiliprotein domain comprises a hapten or an antigen.

36. The complex of claim 20, wherein the complex comprises a fusion protein containing a phycobiliprotein domain which is bound to a cell, virus, subcellular organelle, prion or biological surface.

37. An isolated and purified fusion protein comprising a domain corresponding to a molecule of at least 2000 daltons molecular weight and a domain corresponding to a phycobiliprotein subunit.

38. The fusion protein of claim 37, wherein the molecule of at least 2000 daltons molecular weight is not found associated with phycobiliprotein in algae.

39. The fusion protein of claim 37, wherein the molecule of at least 2000 daltons molecular weight is not found naturally in algae.

40. A method of producing an isolated and purified fusion protein containing a phycobiliprotein domain and a non-phycobiliprotein domain, said method comprising
(1) culturing a recombinant cell transformed with a nucleic acid sequence encoding a phycobiliprotein subunit in reading frame with a nucleic acid sequence encoding a polypeptide domain so that the nucleic acid sequences are expressed as a fusion protein; and
(2) isolating said fusion protein so that it is separated from all other fluorescent moieties in the cell other than fluorescent moieties which may be part of a spontaneously forming complex containing the fusion protein.

41. The method of claim 40, wherein the recombinant cell is an algal cell, and the nucleic acid sequence encoding the non-phycobiliprotein domain is foreign to the algal cell.

42. In a method for screening a combinatorial library to determine moieties which recognize a specific target molecule, the improvement comprising using a detectable label which is a fusion protein containing a phycobiliprotein domain and another domain corresponding to the one member of a random peptide library,
wherein said labeled library is used to locate peptide-containing domains which bind to the target molecule.

43. A method of producing an isolated and purified fusion protein containing a phycobiliprotein and phycobiliprotein linker domain and a nonphycobiliprotein molecule domain, said method comprising
(1) culturing a recombinant cell transformed with a nucleic acid sequence encoding a phycobiliprotein linker in reading frame with a nucleic acid sequence encoding a polypeptide domain so that the nucleic acid sequences are expressed as a fusion protein, wherein said recombinant cell is not an algal cell;
(2) isolating said fusion protein,
(3) mixing said fusion protein with a plurality of phycobiliprotein subunits or phycobilisome subcomplexes missing the phycobiliprotein linker used in production of the fusion protein, and
(4) allowing these to form a complex with the phycobiliprotein linker fusion protein fitting into its proper position in the assembled phycobiliprotein or phycobilisome and/or subunits thereof.

44. An isolated and purified fusion protein comprising a domain corresponding to a nucleic acid binding domain and a domain corresponding to a phycobiliprotein and/or phycobiliprotein linker subunit.

45. An assay method using the fusion protein or protein complex of claim 44, wherein a synthetic oligonucleotide, having a recognition sequence for the nucleic acid binding domain in the fusion protein, binds to the reagent to form a non-covalent complex containing oligonucleotide bound to fluorescent label.

46. The method of claim 45, wherein said complex is allowed to hybridize to target nucleic acid complementary to said oligonucleotide such that hybridization of said target may be detected.
